(19) United States
(12) Patent Application Publication
(10) Pub. No.: US 2005/0137445 A1
(43) Pub. Date: Jun. 23, 2005

(54) Eye massager for receiving magnetic devices

(76) Inventors: Pao-Sheng Chang, Taipei (TW); Ching-Chuan Chang, Taipei (TW)

Correspondence Address: CHIH-CHING HSIEN, 235 CHUNG-HO BOX 8-24, TAIPEI HSIEN, 235, TW

(21) Appl. No.: 10/739,953

(22) Filed: Dec. 19, 2003

(51) Int. Cl. A61N 1/00; A61H 1/00; A61H 1/02; A61H 5/00

(52) U.S. Cl. 600/15; 601/15

(57) ABSTRACT

An eye massager comprises a pair of magnetic devices; and an eye mask. The eye mask comprises a mask frame and a pair of buckles for combining an adjustable tension strip. A front end of the mask frame is a soft mask body. The mask body is formed with two through holes at positions corresponding to those of user's eyes. Each through hole is passed through by and fixed with one magnetic device. Each of two sides of the eye mask is installed with one of the pair of buckles. The mask body and the mask frame are made of hard materials. A periphery of each through hole of the mask body is enclosed by a rubber ring. The magnetic device comprises a permanent magnet; a handle having a receiving portion for receiving the permanent magnet; and a plug for sealing the permanent magnet in the handle.

### FIELD OF THE INVENTION

The present invention relates to eye masks, and particularly to an eye massager which comprises a pair of magnetic devices; and an eye mask. The eye mask comprises a mask frame and a pair of buckles for combining an adjustable tension strip.

### BACKGROUND OF THE INVENTION

In the prior art, the eye massager is mainly formed by a massage bar. One end of the bar is an opening. An interior of the massage bar is formed with a receiving portion for receiving a permanent magnet. A plug serves to seal the receiving portion from the opening. A top of the plug is a groove and a bottom of the plug is a hole. Magnetic fields generated from the permanent magnet will pass through the bottom planes and the grooves of the plugs to active the eye's nerves so as to adjust the function of eyes.

However the defect of the prior art eye massager is that the user must holds the massage bar to touch and press the periphery of the eyes. For a longer time, the user's hand will feel ache. Thereby, it is often that the user dose not use the massage bar over a long period of time.

### SUMMARY OF THE INVENTION

Accordingly, the primary object of the present invention is to provide an eye massager which can be used easily and conveniently.

To achieve above objects, the present invention provides an eye massager which comprises a pair of magnetic devices; and an eye mask. The eye mask comprises a mask frame and a pair of buckles for combining an adjustable tension strip. A front end of the mask frame is a mask body made of soft material. The mask body is formed with two through holes at positions corresponding to those of eyes of users. Each through hole is passed through and fixed with one magnetic device. Each of two sides of the eye mask is installed with one of the pair of buckles. The mask body and the mask frame are made of hard materials. Each through hole of the mask body is enclosed by a rubber ring. The magnetic device comprises a permanent magnet; a handle having a receiving portion for receiving the permanent magnet; and a plug for sealing the permanent magnet in the handle.

The various objects and advantages of the present invention will be more readily understood from the following detailed description when read in conjunction with the appended drawing.

### BRIEF DESCRIPTION OF THE DRAWINGS

FIG. 1 is a perspective view of the eye massager of the present invention.

FIG. 2 is an upper schematic cross section view of the eye massager of the present invention.

FIG. 3 is a lateral cross section view of the eye massager of the present invention.

FIG. 4 is a schematic view showing the magnetic device of the eye massager of the present invention.

FIG. 5 is an exploded perspective view of the magnetic device of the eye massager of the present invention.

FIG. 6 is a structural perspective view of the eye mask of the present invention.

### DETAILED DESCRIPTION OF THE PREFERRED EMBODIMENTS

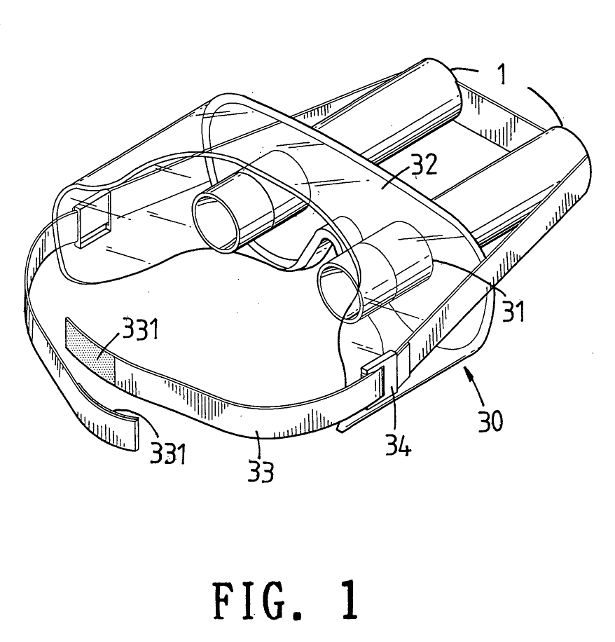

With reference to FIG. 1, the eye massager of the present invention is illustrated. The eye massager includes an eye mask 30 and a magnetic device 1.

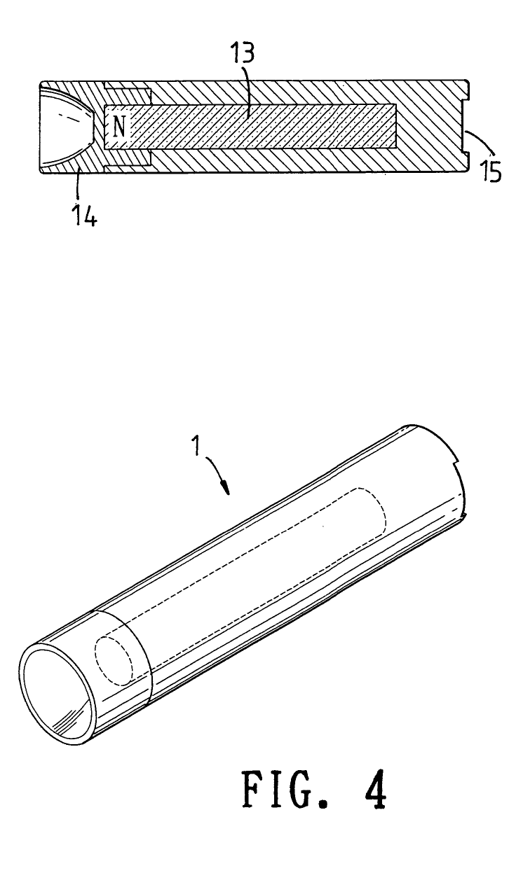

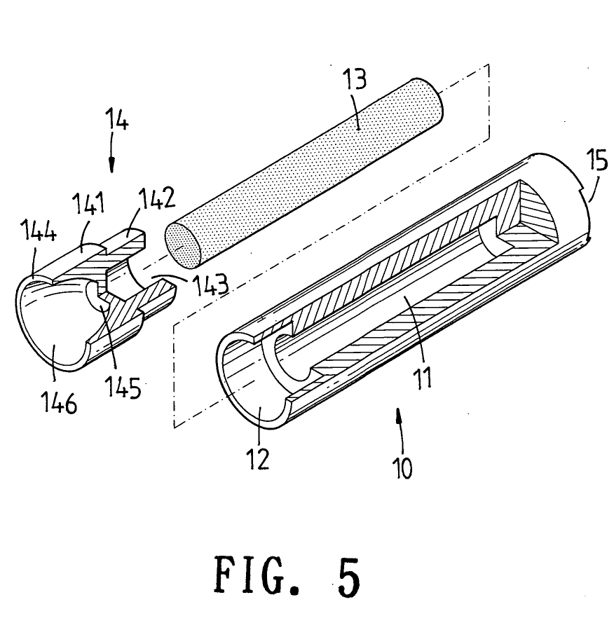

Referring to FIGS. 4 and 5, the magnetic device 1 has a plug 10, a permanent magnet 13 and a handle 10.

The permanent magnet 13 is a long cylinder with preferred permeability. When the permanent magnet 13 is induced by a strong magnetic field, it will generate a strong magnetic force.

An upper portion 141 of the plug 14 has a larger diameter than a lower portion 142 thereof. A lower portion 142 is formed with a hole 142 having a diameter approximately equal to that of the permanent magnet 13. The upper portion 141 of the plug 14 is formed with a groove 146. A bottom surface of the groove 146 is a plane 145. An opening of the groove 146 is formed with an annular lip 144.

One end of the handle 10 is formed with a concave portion 15. The interior of the handle 10 is formed with a receiving portion 11 having an opening 12 at another end of the handle 10 for receiving the permanent magnet 13. The plug 14 covers upon the opening 12 for sealing the receiving portion 11. If necessary, the plug 14 can be stuck or welded to the handle 10 by following a surface process so as to firmly fix the plug 14 to the handle 10.

The eye mask 30 has a mask frame 36. A front end of the mask frame 36 is a mask body 32 made of soft material. The mask body 32 is formed with two through holes 31 at positions corresponding to those of eyes of users. Each through hole 31 is passed through by and fixed with the magnetic device 1 of the present invention. Each of two sides of the eye mask 30 is installed with a buckle for combining an adjustable tension strip 33.

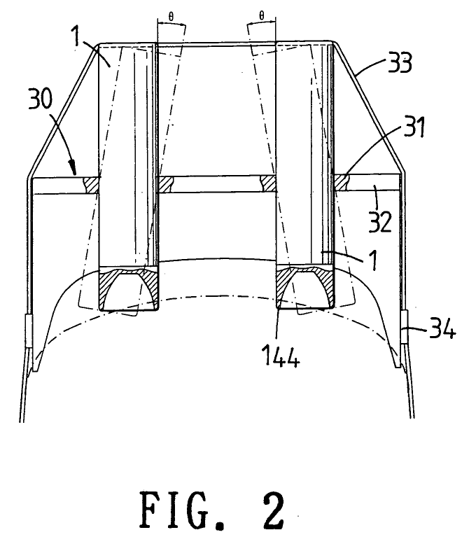

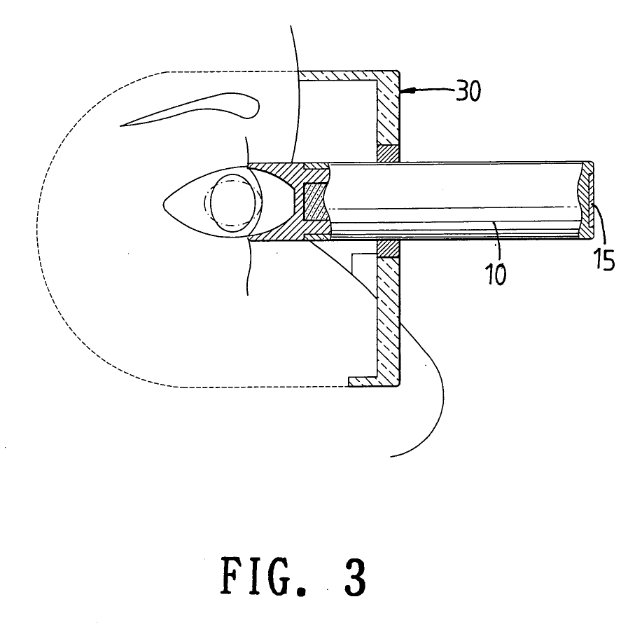

Referring to FIGS. 2 and 3, and FIGS. 1 and 5, in operation, the adjustable tension strip 33 is buckled with the concave portions 15 of the magnetic devices 1. Then the eye mask 30 is worn on the head of the user and the magnetic devices 1 are placed upon the eyes of the user. After the eye mask 30 is adjusted to a preferred position, the two ends 331 of the adjustable tension strip 33 are combined. Thereby, the annular lips 144 of the magnetic devices 1 will apply pressures to the eyes and the magnetic fields generated from the permanent magnet 13 will pass through the bottom planes 145 and the grooves 146 of the plug 14 to activate the eye's nerves so as to adjust the function of eyes.

Moreover, in the present invention, the permanent magnet 13 can rotate through a small angle. If necessary, the permanent magnet 13 can move up and down, right or left so as to massage the muscles and improve the eye ability.

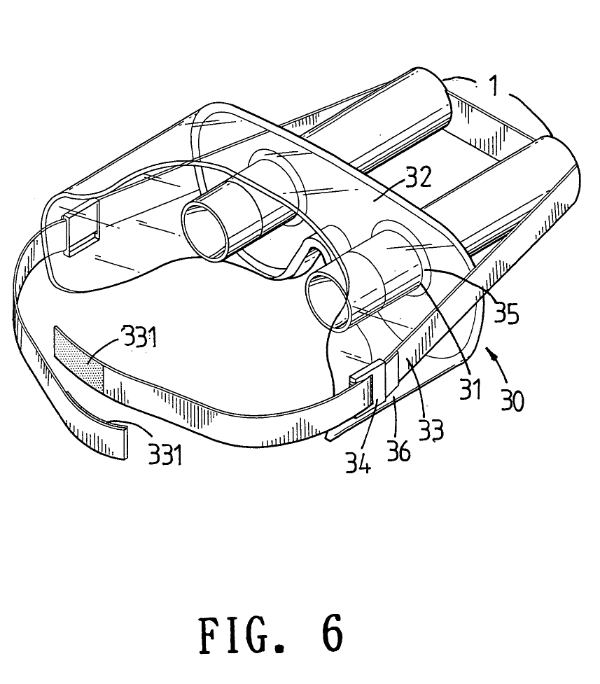

Moreover, in another embodiment of the present invention, referring to FIG. 6, the mask frame 36 and mask body 32 are made of hard material. A rubber ring 35 is installed around a periphery of through hole 31 of the mask body 32, which achieves the same function as above said embodiment.

Thereby, the eye massager of the present invention improves the defect of prior art which must be adjusted by hands. By the magnetic device of the present invention, it is only necessary to enclose the eye mask upon the head of the user. It is unnecessary to operate the eye massager by one hand with a great force. Moreover, the magnetic device can rotate within a small angle. Thereby, the prior art effect will be achieved by the present invention. Moreover, the use of the present invention is easy and convenient with a good treating effect.

The present invention is thus described, it will be obvious that the same may be varied in many ways. Such variations are not to be regarded as a departure from the spirit and scope of the present invention, and all such modifications as would be obvious to one skilled in the art are intended to be included within the scope of the following claims.

1. An eye massager comprising
a pair of magnetic devices; and
an eye mask comprising:
a mask frame; a front end of the mask frame being a soft mask body; the mask body being formed with two through holes at positions corresponding to those of eyes of users; each through hole being passed through by and fixed with one of the magnetic devices;
a pair of buckles; each of two sides of the eye mask being installed with one of the pair of buckles for combining an adjustable tension strip.

2. The eye massager as claimed in claim 1, wherein the mask body and the mask frame are made of hard materials.

3. The eye massager as claimed in claim 1, wherein a periphery of each through hole of the mask body is enclosed by a rubber ring.

4. The eye massager as claimed in claim 1, wherein the magnetic device containing a magnetic device.

5. The eye mask as claimed in claim 1, wherein the magnetic device comprising:
a permanent magnet;
a handle having a receiving portion for receiving the permanent magnet; and
a plug for sealing the permanent magnet in the handle.
